(19) United States
(12) Patent Application Publication
(10) Pub. No.: US 2006/0108219 A1
(43) Pub. Date: May 25, 2006

(54) Chemical sensor and chemical sensor apparatus

(75) Inventors: Ryo Kuroda, Tokyo (JP); Tomohiro Yamada, Tokyo (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 10/530,182

(22) PCT Filed: Jun. 23, 2004
(86) PCT No.: PCT/JP04/09239

§ 371 Date: Aug. 22, 2005

(30) Foreign Application Priority Data
Jun. 23, 2003 (JP) 2003177911

(51) Int. Cl. C12Q 1/00

(52) U.S. Cl. 204/403.1; 257/414

(57) ABSTRACT

A chemical sensor for detecting a reaction of a sensor material with a specimen on the basis of an intensity of a surface plasmon polariton wave propagated along a surface of a sensor medium including the sensor material is principally constituted by the sensor medium. The sensor medium includes a periodic structure and the sensor material disposed on the periodic structure. The periodic structure has a pitch substantially equal to an integral multiple of a wavelength of the surface plasma polariton wave generated by irradiating an interface between the periodic structure and the sensor material with light.

### TECHNICAL FIELD

The present invention relates to a chemical sensor and a chemical sensor apparatus, and particularly relates to a chemical sensor and a chemical sensor apparatus including a biosensor for use in medical cave, a health examination, food evaluation, etc.

### BACKGROUND ART

In recent years, demands on medical diagnostics, food evaluation, etc., have been further increased, and development of a compact, high-speed sensing, and an inexpensive biosensor has been required. For this reason, a biosensor for effecting an electrochemical detection using an electrode or an FET (field-effect transistor) has been developed.

Further, a sensor which realize further integration and low production cost and can be used in any measurement environment, is required. As the sensor, a biosensor using surface plasmon resonance as a transducer is most promising. The biosensor detects whether there arises adsorption of a substance, such as an antigen in an antigen-antibody reaction, by using the surface plasmon resonance generated in a thin metal film disposed on a surface of a total (internal) reflection-type prism.

Further, an optical transmission apparatus or the like wherein a metal film is provided with a periodic opening (aperture) or a periodically changing surface shape to enhance optical transmission through the opening or surface shape has been conventionally proposed as described in U.S. Pat. Nos. 5,973,316; 6,052,238; and 6,236,033.

However, in the above described surface plasmon resonance sensor, various trials for realization of high sensitivity, such as reduction in noise of a light source and detection signal processing, have been made. However, as medical care, a health examination or food evaluation becomes a higher level, a further high-sensitive chemical sensor is required. As a result, realization of high sensitivity has been approaching its limit.

### DISCLOSURE OF THE INVENTION

An object of the present invention is to provide a chemical sensor capable of further improving a sensor sensitivity.

Another object of the present invention is to provide a chemical sensor apparatus including the chemical sensor.

The present invention provides chemical sensors and chemical sensor apparatuses which are constituted as follows.

More specifically, according to the present invention, there is provided a chemical sensor for detecting a reaction of a sensor material with a specimen on the basis of an intensity of a surface plasmon polariton wave propagated along a surface of a sensor medium comprising the sensor material, the chemical sensor comprising the sensor medium,

wherein the sensor medium comprises a periodic structure and the sensor material disposed on the periodic structure, the periodic structure having a pitch substantially equal to an integral multiple of a wavelength of the surface plasma polariton wave generated by irradiating an interface between the periodic structure and the sensor material with light.

As described above, the present invention is characterized in that the period of the periodic structure is substantially identical to an integral multiple of a wavelength of the surface plasmon polariton wave (hereinafter referred to as the “SPP wave”) generated at an interface between the periodic structure and the sensor material. As the periodic structure, for example, it is possible to use a small opening array or a small uneven structure array disposed on the thin metal film described above or a fine metal particles disposed on a substrate. It becomes possible to realize a high-sensitivity chemical sensor by causing light to enter the periodic structure and detecting its-transmitted light or its reflected light.

For example, in the case of the small openings formed on the thin metal film, the SPP waves propagated along an upper surface of the thin metal film in an in-plane mode have the same phase resonance-enhancedly to increase their amplitudes, thus being concentrated at the small openings. Accordingly, the transmitted light generated by scattering of the incident light at a lower portion of the small openings principally comprises a component attributable to the SPP waves propagated along the upper surface of the thin metal film in an in-plane mode over a longer distance, compared with a component of light directly incident on the small openings and transmitted therethrough. The SPP waves are accompanied with a change in dielectric constant or thickness of the sensor material, i.e., change in wavelength depending on a degree of a reaction of the sensor material with the specimen, at the time of propagation through the interface between the thin metal film and the sensor material. Accordingly, it is possible to selectively detect the component attributable to the SPP waves having information on the degree of a reaction of the sensor material with the specimen by detecting the above described transmitted light. As a result, it becomes possible to realize detection at a high sensitivity.

Further, for example, by providing a length of circumference (circumferential length) of a thin metal film portion between adjacent two openings so as to be substantially an integral multiple of wavelength of the SPP wave, an SPP wave which is once propagated on the lower surface side of a thin metal film portion after being passed through a small opening and then returned to the upper surface side of the thin metal film portion through an adjacent small opening is in phase with an SPP wave propagated along the upper surface of the thin metal film portion in an in-plane mode, so that a height of resonance peak in a transmitted light intensity spectrum is further increased but a width thereof becomes narrower. As a result, an amount of a change in transmitted light quantity with respect to a change in position of resonance peak due to a reaction of the sensor material with the specimen becomes larger, thus further improving a sensitivity of the sensor.

In the present invention, it is possible to adopt the following constitutions.

In the chemical sensor of the present invention, the sensor material may be a biochemical sensor material.

In the chemical sensor, the periodic structure may comprise a plurality of openings provided in a metal film with a predetermined pitch, the openings having a size smaller than a wavelength of the irradiation light.

In the chemical sensor, the openings may have a substantially circular shape or a substantially polygonal shape, and their periodic arrangement may be a two-dimensional arrangement in the metal film surface.

In the chemical sensor, the openings may have a slit shape, and their periodic arrangement may be a one-dimensional arrangement in the metal film surface.

In the chemical sensor, the openings having a slit-like shape may include adjacent two openings sandwiching a metal film portion having a length of circumference which is a substantially integral multiple of a wavelength of the surface plasmon polariton wave.

In the chemical sensor, the periodic structure comprising a plurality of openings provided in a metal film with a predetermined pitch may be provided in a plurality of periodic structures which have the same or different sizes and/or pitches of their openings and the same or different arrangement directions.

In the chemical sensor, the periodic structure may comprise at least one opening provided in a metal film with a predetermined pitch and at least one recess portion or projection portion provided in the metal film, and the opening may have a size which is smaller than a wavelength of the irradiation light.

In the chemical sensor, the opening and the recess portion or the projection portion may have a substantially circular shape or a substantially polygonal shape, and their periodic arrangements may be a two-dimensional arrangement.

In the chemical sensor, the two-dimensional arrangement may be such an arrangement that the recess portion or the projection portion is disposed concentrically around the opening.

In the chemical sensor, the opening and the recess portion or the projection portion may have a slit-like shape, and their periodic arrangements may be a one-dimensional arrangement.

In the chemical sensor, the opening may include adjacent two openings sandwiching a metal film portion having a length of circumference which is a substantially integral multiple of a wavelength of the surface plasmon polariton wave.

In the chemical sensor, the metal film may be a film of a metal or alloy selected from the group consisting of gold, silver, copper, and aluminum.

In the chemical sensor, the periodic structure may comprise fine metal particles disposed on a substrate with a predetermined pitch, and the fine metal particles may have a size which is smaller than a wavelength of the surface plasmon polariton wave.

In the chemical sensor, the fine metal particles may have a length of circumference which is a substantially integral multiple of a wavelength of the surface plasmon polariton wave.

In the chemical sensor, the sensor medium may comprise the periodic structure and a substrate for the sensor material disposed on the periodic structure, and the substrate may comprise a prism.

According to the present invention, there is also provided a sensor apparatus including: any one of the above described chemical sensors, a light source for irradiating the chemical sensor with light, and a photodetector for detecting light transmitted through or reflected from the chemical sensor.

In the chemical sensor apparatus, the photodetector may comprise a spectroscope.

In the chemical sensor apparatus, the photodetector may comprise means for detecting light transmitted through a band-pass filter.

In the chemical sensor apparatus, the sensor medium may be integrally supported in a micro total analysis system prepared through a semiconductor process.

In the chemical sensor apparatus, the sensor medium may be integrally supported in a DNA chip prepared through a semiconductor process.

In the chemical sensor apparatus, the sensor medium may be integrally supported in a protein chip prepared through a semiconductor process.

These and other objects, features and advantages of the present invention will become more apparent upon a consideration of the following description of the preferred embodiments of the present invention taken in conjunction with the accompanying drawings.

### BRIEF DESCRIPTION OF THE DRAWINGS

FIG. 1 is a sectional view showing a structure of a sensor medium used in a chemical sensor apparatus according to an embodiment of the present invention.

FIGS. 2(a) and 2(b) are respectively a view showing an example of a periodic arrangement of small openings in the sensor medium in the embodiment of the present invention, wherein FIG. 2(a) shows an example in which an array of small slit openings is periodically arranged in an X direction in a thin metal film with each short side in the x direction and each long side in a y direction, and FIG. 2(b) shows an example in which a two dimensional array of small openings are arranged in x and y directions, respectively, in a thin metal film.

FIG. 3 is a view showing an example of a transmitted light intensity spectrum in the embodiment of the present invention.

FIG. 4 is a view showing a propagation passage of SPP wave moving around a thin metal film portion through adjacent small openings in the embodiment of the present invention.

FIG. 5 is a view showing a structure of a chemical sensor apparatus using a sensor medium in Embodiment 1 of the present invention.

FIGS. 6(a) to 6(g) are views for illustrating a process for producing the sensor medium in Embodiment 1 of the present invention.

FIGS. 7(a) to 7(e) are views for illustrating a nanomolding process as the production process of the sensor medium in Embodiment 1 of the present invention.

FIGS. 8(a), 8(b) and 8(c) are views showing a shape of a pattern array provided on a thin metal film of a sensor medium in EMbodiment 2 of the present invention, wherein FIG. 8(a) is a sectional view and FIGS. 8(b) and 8(c) are top views.

FIGS. 9(a) and 9(b) are views showing a shape of a pattern array provided on a thin metal film of a sensor medium in Embodiment 3 of the present invention, wherein FIG. 9(a) is a sectional view and FIG. 9(b) is a top view.

FIG. 10 is a view showing a structure of a sensor medium using a prism as a substrate in Embodiment 4 of the present invention.

FIG. 11 is a view showing a structure of a multi-channel sensor medium comprising a plurality of slits and two-dimensional small openings in a thin metal film in Embodiment 5 of the present invention.

FIG. 12 is a view showing a structure of a multi-channel chemical sensor apparatus including the multi-channel sensor medium shown in FIG. 11.

FIG. 13 is a view showing a structure in which a sensor medium is integrally formed into a micro total analysis system according to an example of Embodiment 6 of the present invention.

FIG. 14 is a view showing a structure in which a sensor medium is integrally formed into a DNA chip or a protein chip according to another example of Embodiment 6 of the present invention.

### BEST MODE FOR CARRYING OUT THE INVENTION

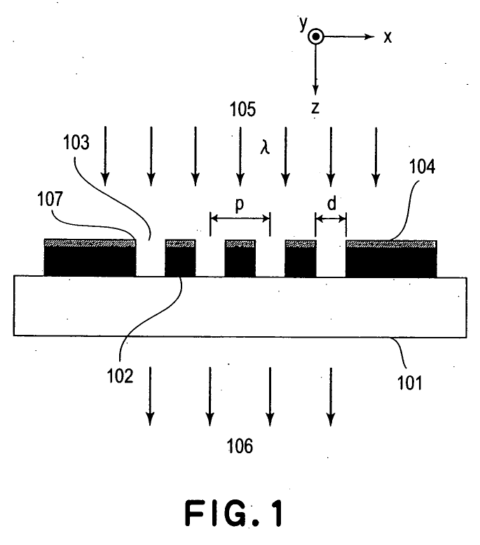

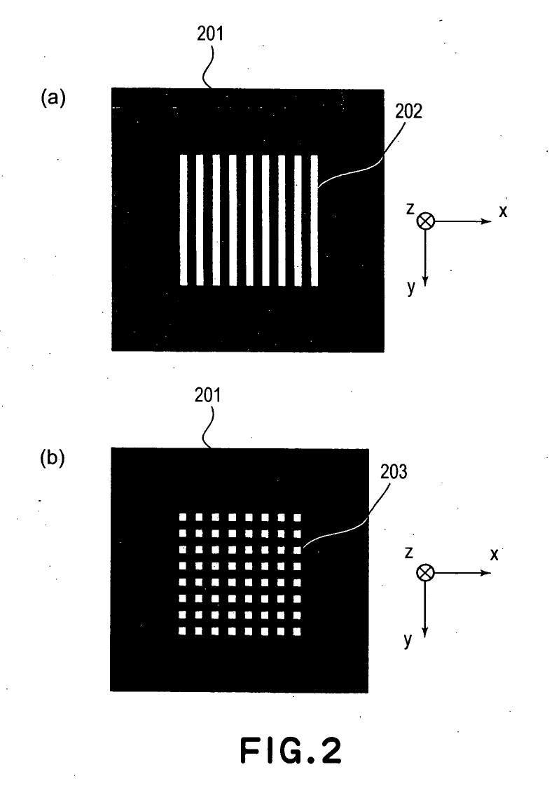

Hereinbelow, embodiments of a sensor medium of the chemical sensor apparatus according to the present invention will be described with reference to FIGS. 1 and 2.

The chemical sensor apparatus includes a sensor material which contacts a specimen (to be tested) to be reacted with a substance to be detected, and a measuring device for measuring a change in sensor material before and after the contact through an optical method or the like. In some cases, the sensor material is separated from the measuring device portion and is subjected to the reaction with the specimen, so that it is held on an appropriate support. Such a structure is referred herein to as a “sensor medium”.

FIG. 1 is a sectional view showing a structure of a sensor medium of a chemical sensor apparatus according to this embodiment of the present invention.

Referring to FIG. 1, the sensor medium of this embodiment includes a transparent substrate 101, a thin metal film 102, a small opening 103, and a sensor material 104. On the transparent substrate 101, the thin metal film 102 is disposed with a plurality of small openings 103. On the thin metal film 102, the sensor material 104 is disposed. The thin metal film 102 is provided with the small openings 103 arranged periodically in one direction of the thin metal film 102 surface, i.e., in an x direction shown in FIG. 1. The thin metal film 102 has a thickness of about 10-500 nm.

FIGS. 2(a) and 2(b) are respectively a view of the periodically arranged small openings 103 as seen from above.

FIG. 2(a) shows an example of the periodically arranged small openings formed by periodically arranging an array 202 of small slit openings win an x direction in a thin metal film 201 with each short side in the x direction and each long side in a y direction. Each of the small openings 103 is a slit-like opening having a length, in the y direction perpendicular to the x direction, which is larger than a width in the x direction.

Further, FIG. 2(b) shows an example of the periodically arranged small openings formed by periodically arranging a two dimensional array 203 of small openings both in the x direction and the y direction in the thin metal film 201.

A width d of the small openings 103 in the x direction (a width of the array 202 in the x direction, a width of the two-dimensional array 203 in the x direction, and a width (length) of the two-dimensional array 203 in the y direction) is a value selected in a range of 1-200 nm, which is smaller than a wavelength of irradiated light described later.

Further, a pitch of the small openings has a value substantially equal to a wavelength of a SPP wave generated at the time of irradiating an interface between the thin metal film 102 and the sensor material 104.

The SPP wave has a wavelength λsp represented by the following equation (1):
λsp=λ{(εm+εs)/(εm−εs)}^{1/2} (1)
wherein λ represents a wavelength of irradiated light, εm represents a dielectric constant of the thin metal film, and εs represents an effective dielectric constant of the sensor material as seen from the interface with the thin metal film.

On the thin metal film 102, the sensor material 104 is disposed. The sensor material 104 fixes an objective substance to be detected, contained in the specimen, by surface adsorption or chemical bonding when the sensor medium contacts the specimen. Alternatively, the sensor material may be a material constituted by components containing a component which is bonded to the objective substance to be detected and then is dissociated from the constitutional components. In this embodiment, the sensor material comprises a material changed in its dielectric constant (refractive index or absorptivity) with respect to light by the bonding thereof to the objective substance to be detected or the dissociation of constitutional component.

A principle of operation of the chemical sensor of the present invention will be described with reference to FIGS. 1, 3 and 4.

Referring to FIG. 1, incident light 10 is caused to enter the arrangement of the small openings 103 in a downward direction (+z direction) in the drawing. At this time, the incident light is scattered at a corner 107 of the thin metal film 102 facing the small opening 103, whereby an SPP wave is excited at an interface between the thin metal film 102 and the sensor material 104 and is propagated toward a periphery of the thin metal film 102 at an upper surface of the thin metal film 102 in an in-plane mode. At that time, when the pitch P of the periodically arranged small openings 103 coincides with an integral multiple of a wavelength of the SPP wave, the SPP wave is excited at respective corners of the thin metal film 102. The resultant SPP waves propagated in the upper plane of the thin metal film 102 have the same phase to increase their amplitude. As a result, of these SPP waves, a component which rounds the corner and enters the small opening 103 and a component which excites an SPP wave propagated in a lower plane of the thin metal film 102 by a downward escaping of near-field light are increased, so that a light amount of transmitted light 106 generated by scattering of these SPP waves on the lower surface side of the small openings 103.

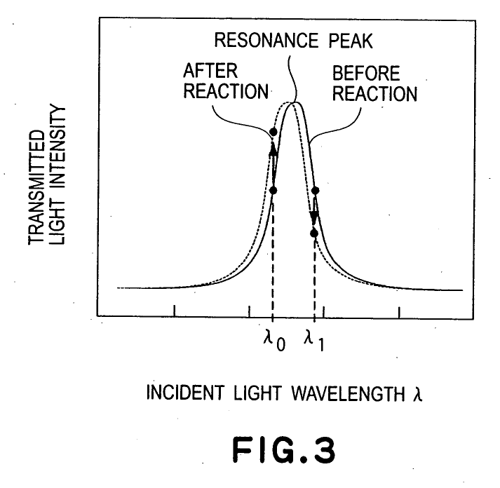

An example of a transmitted light intensity spectrum of the sensor material used in this embodiment is shown in FIG. 3 by a solid line which represents the spectrum before reaction of the sensor material. The sensor medium shown in FIG. 1 is caused to contact the specimen to be tested, such as specimen liquid or specimen gas,. thus causing a reaction of the sensor material with the specimen. A transmission spectrum after the reaction is shown in FIG. 3 by a dotted line.

As shown in FIG. 3, the spectrum is changed by the reaction. This is because an effective dielectric constant εs as seen from the boundary surface of the sensor material 104 with the thin metal film 102 is changed by a change in thickness of the film of the sensor material 104 on the thin metal film 102 or a change in dielectric constant of the sensor material 104 per se due to the reaction between the sensor material 104 and the specimen. For this reason, as apparent from the above described equation (1), the wavelength λsp of the excited SPP wave is changed. By this change, the position of a resonance peak of the transmitted light intensity spectrum is changed as shown in FIG. 3 as an example.

Such a change in position of the resonance peak may be determined by, e.g., causing light with a board wavelength spectrum width to enter the sensor medium as incident light and detecting a spectrum shape through a spectroscope. Further, it is also possible to detect a change in transmitted light intensity as indicated by a left-hand (upward) arrow in FIG. 3 by irradiating the sensor medium with monochromatic light with a wavelength somewhat shifted from a center wavelength of resonance peak, i.e., the wavelength of SPP wave, as shown by a wavelength λ0 in FIG. 3. In the latter case, a detection apparatus becomes compact since the need for the spectroscope is eliminated.

Examples of the sensor material causing the above described reaction may include:

(1) an antibody substance which causes a specific bonding to an antigen substance contained in the specimen, and

(2) a complex of an enzyme with an analogous substance, to be measured, contained in the specimen, which complex being dissociated by contact of an antigen as a substance to be measured with the complex to form an antigen-antibody complex between the enzyme and the substance to be measured.

Such a sensor material may be those causing a change in effective dielectric constant when viewed from the interface of the sensor material with the thin metal film, such as a film thickness change or a refractive index change, through the reaction between the sensor material and the specimen, and may include materials which can be used in a chemical sensor including biosensors, such as an enzyme sensor, a microbial sensor, an organella sensor, a tissue sensor, an immunosensor, an enzyme immunosensor, and a biochemical affinity sensor.

In the above described structure of the sensor medium, the SPP waves propagated at an upper surface of the thin metal film in an in-plane mode have the same phase resonance-enhancedly to increase their amplitudes, thus being concentrated at the small openings. Accordingly, the transmitted light generated by scattering of the incident light at a lower portion of the small openings principally comprises a component attributable to the SPP waves propagated along the upper surface of the thin metal film in an in-plane mode over a longer distance, compared with a component of light directly incident on the small openings and transmitted therealong. The SPP waves are accompanied with a change in dielectric constant or thickness of the sensor material, i.e., change in wavelength depending on a degree of a reaction of the sensor material with the specimen, at the time of propagation along the interface between the thin metal film and the sensor material. Accordingly, it is possible to selectively detect the component attributable to the SPP waves having information on the degree of a reaction of the sensor material with the specimen by detecting the above described transmitted light. As a result, it becomes possible to realize detection at a high sensitivity.

Further, in the case where a circumferential length of a thin metal film portion between adjacent two openings is a substantially integral multiple of wavelength of the SPP wave, an SPP wave which is once propagated on the lower surface side of a thin metal film portion after being passed through a small opening and then returned to the upper surface side of the thin metal film portion through an adjacent small opening is in phase with an SPP wave propagated at the upper surface of the thin metal film portion in an in-plane mode, so that a height of resonance peak in a transmitted light intensity spectrum shown in FIG. 3 as an example is further increased but a width thereof becomes narrower. As a result, an amount of a change in transmitted light quantity with respect to a change in position of resonance peak due to a reaction of the sensor material with the specimen becomes larger, thus further improving a sensitivity of the sensor.

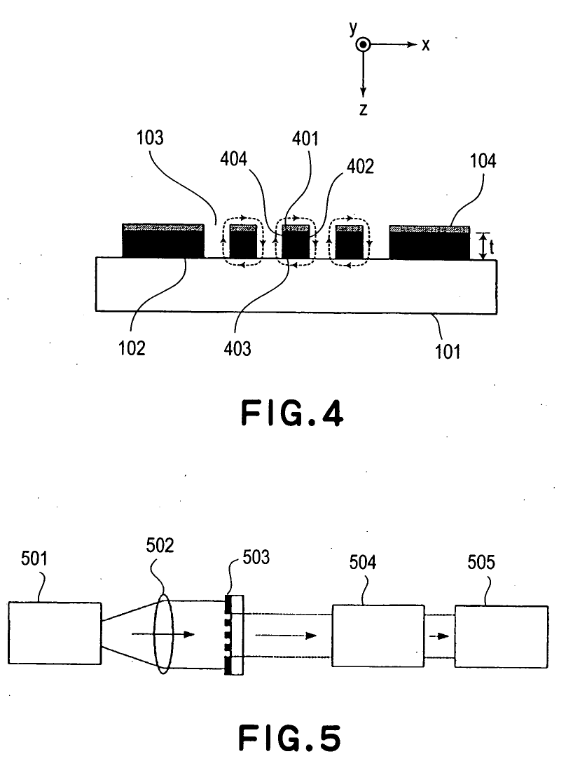

Next, the above described circumferential length will be described with reference to FIG. 4.

Referring to FIG. 4, a propagation path of SPP wave which is propagated around a thin metal film portion through adjacent small openings, is indicated by a dotted line with arrows.

In FIG. 4, such a state that the SPP wave is propagated around the thin metal film portion in a clockwise direction indicated by the arrows is shown. However, there is similarly present an SPP wave which is propagated in a counterclockwise direction.

Herein, the circumferential length (the length of circumference) of the SPP wave can be obtained by the sum of a length of a portion A401 which is an interface between an upper (top) surface of the thin metal film 102 and the sensor material 104, a length of portion B402 corresponding to a propagation length in the small opening 103, a length of a portion D404 corresponding to a propagation length in the small opening 103, and a length of a portion C403 which is an interface between a lower (bottom) surface of the thin metal film 102 and the transparent substrate 101.

Of these lengths, effective lengths of the portion A401 and the portion C403 can be determined from the above described equation (1) by substituting dielectric constant values of the sensor material and the transparent substrate material for εs, respectively, in the equation (1), while taking their layer (film) thicknesses into consideration.

Further, effective lengths of the portion B402 and the portion D404 can be determined on the basis of the material for the thin metal film and the shape of the small openings (a width of opening and a thickness of the thin metal film).

As described above, the sum of these lengths of the portions A, B, C and D (401, 402, 403 and 404) is defined herein as a circumferential length.

In order to provide the circumferential length which is substantially equal to an integral multiple of the wavelength of SPP wave, a thickness t of the thin metal film may be adjusted.

In the above description, the small slit opening array and the two-dimensional small opening array shown in FIG. 2 are described, as an example, as a structure for exciting an SPP wave and propagating the SPP wave at the surface of the thin metal film in an in-plane mode. However, in the present invention, it is possible to use an uneven array which does not penetrate the thin metal film, in place of the opening arrays. Further, it is also possible to use a fine metal particle array which has an inverse structure of the two-dimensional small opening array.

Further, in the above description, detection of light transmitted through the small openings is described as an example but in the present invention, detection of light which is incident on the small opening and reflected therefrom may be performed. However, compared with the case of detecting the reflected light, the detection of transmitted light passing through the small opening is advantageous for improvement in S/N (signal-to-noise) ratio of signal strength since it is less affected adversely by stray light.

Hereinbelow, specific embodiments of the chemical sensor apparatus according to the present invention and its array shape and so on will be described.

### Embodiment 1

FIG. 5 shows a structure of a chemical sensor apparatus using a sensor medium in this embodiment according to the present invention.

Referring to FIG. 5, the chemical sensor apparatus includes a tungsten lump 501 a collimator lens 502, a sensor medium 503, and a spectroscope 504. In the figure, while light emitted from the tungsten lump 501 is changed to substantially parallel light by the collimator lens 502 to enter the sensor medium 503. Transmitted light passed through the sensor medium 503 is caused to enter the spectroscope 504 to be spectrum-separated and is detected by a multi-channel analyzer 505, whereby spectrum information is obtained.

A material for a thin metal film (102 in FIG. 1) of the sensor medium can be selected from ordinary metals. Particularly, gold, silver, copper, and aluminum provides a large intensity of surface plasmon, thus being suitable in this embodiment. Among these metals, gold has a peak attributable to localized plasmon resonance over the entire visible region, thus being an optimum material for constituting a sensor which performs detection with visible light. Further, by using an alloy, selected from gold, silver, copper, and aluminum, having a predetermined mixing ratio, it is possible to adjust a peak position between a near-ultraviolet region and a near-infrared region.

The sensor material (104 in FIG. 1) may be a material which reacts with the specimen to cause a change in film thickness, a change in refractive index, a change in absorption spectrum, or a change in fluorescence spectrum, and may include materials which can be used in a chemical sensor including biosensors, such as an enzyme sensor, a microbial sensor, an organella sensor, a tissue sensor, an immunosensor, an enzyme immunosensor, and a biochemical affinity sensor.

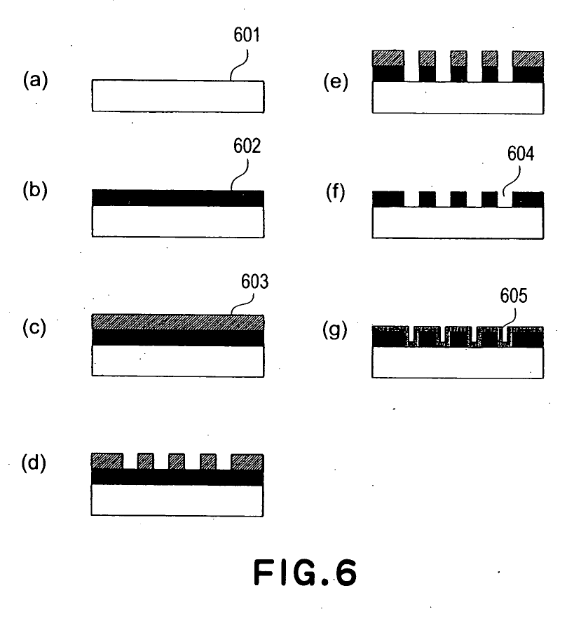

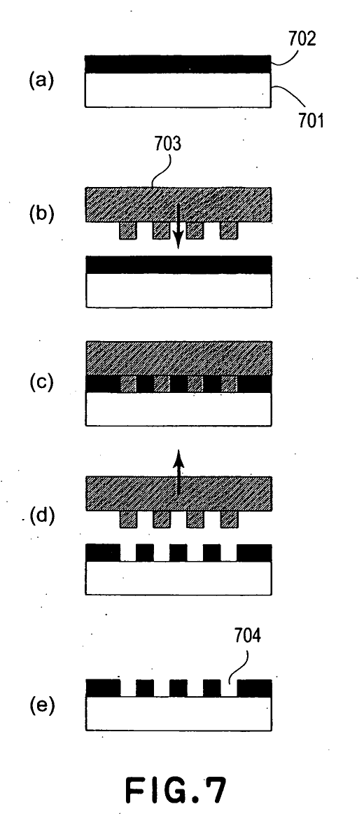

Next, a process for preparing the sensor medium in this embodiment will be described with reference to FIGS. 6 and 7.

First a quartz substrate 601 is prepared (FIG. 6(a)), on the quartz substrate 601, a 50 nm-thick metal film 602 is formed through sputtering (FIG. 6(b)).

Then, on the thin metal film 602, a 10 nm-thick electron beam resist 603 is formed by spin coating (FIG. 6(c)). The result substrate is subjected to exposure by an electron beam irradiation apparatus and then is subjected to development to obtain a resist pattern with a L/S (line/space)=20 nm/20 nm (FIG. 6(d)).

Thereafter, the thin metal film 602 is etched (FIG. 6(e)) to remove the resist, thus providing small openings 604 with an opening width of 20 nm and an opening pitch (spacing) of 20 nm (FIG. 6(f)).

Finally, the thin metal film 602 is surface-treated and a sensor material 605 is bonded to the thin metal film 602 (FIG. 6(g)).

In this embodiment, the small opening pattern is prepared by the electron beam irradiation apparatus but may also be prepared by other apparatuses including a focusing ion beam processing apparatus; various probe processing apparatuses to which principles of a scanning tunneling microscope, an atomic force microscope, and a near-field optical microscope are applied; an X-ray exposure apparatus; EUV (extreme ultraviolet) exposure apparatus; and an electron beam stepper.

Further, when the small opening pattern is prepared by an exposure apparatus using near-field light as described in U.S. Pat. No. 6,171,703, a nanomolding method shown in FIG. 7, or a nanoimprinting method using a thermoplastic resin or a thermosetting resin, it is possible to realize a sensor medium simply and inexpensively.

Next, the preparation process of the sensor medium through the nanomolding method will be described with reference to FIG. 7(a) to 7(e).

On a quartz substrate 701, a 50 nm-thick thin metal film 702 is formed (FIG. 7(a)).

Then, a replica plate 703 having a pattern of L/S (line/space)=20 nm/20 nm is pressed against the surface of the thin metal film 702 under a load (FIGS. 7(b) and 7(c)), and thereafter the replica plate 703 is removed from the substrate (FIG. 7(d)) to form small openings 704 (FIG. 7(e)).

In this embodiment, a shape of an array of the small opening pattern may, e.g., be those of the slit opening array shown in FIG. 2(a) and the two-dimensional small opening array shown in FIG. 2(b). As the two-dimensional small opening array, in addition to one having a rectangular (square) lattice with a pitch, shown in FIG. 2(b), which is substantially equal to a wavelength of SPP wave, it is possible to use one having a triangular lattice with a pitch substantially equal to a wavelength of SPP wave.

### Embodiment 2

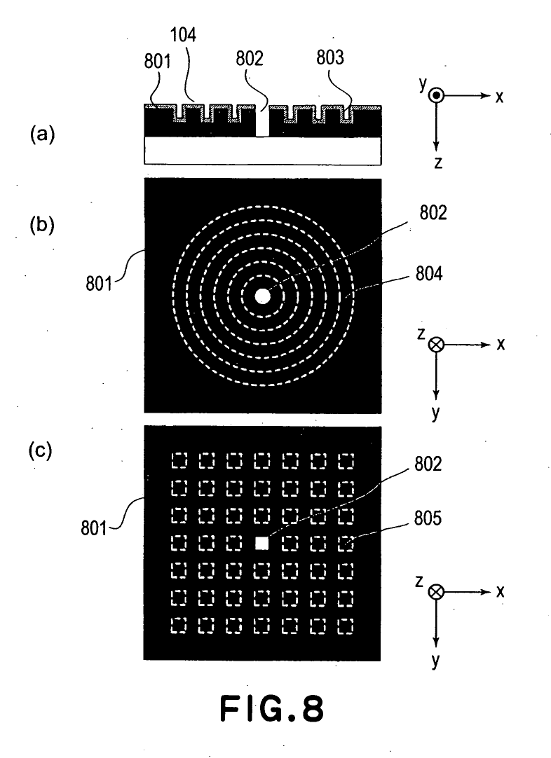

FIGS. 8(a), 8(b) and 8(c) show a structure of a sensor medium having a pattern array shape in this embodiment. FIG. 8(a) is a section view thereof, and FIGS. 8(b) and 8(c) are top views thereof.

As shown in FIG. 8(a), a small opening 802 is provided at a center of a thin metal film 801. In a portion surrounding the center, a small uneven structure array 803 having a pitch (spacing) which is substantially equal to a wavelength of SPP wave, is provided.

The small uneven structure array 803 may be one wherein around the small opening 802, concentric small uneven array 804 is disposed (FIG. 8(b)) or one wherein around the small opening 802, a two-dimensional small uneven array 805 is disposed (FIG. 8(c)).

By disposing the small uneven array around the small opening as described above, it is possible to reduce an influence of direct light which directly passes through the small opening without being propagated along the interface between the thin metal film 801 and the sensor material 104 in the form of SPP wave. Further, the SPP wave propagated along the interface between the thin metal film and the sensor material is concentrated in the small opening and passes through the small opening, so that it becomes possible to improve an S/N ratio for signal detection.

### Embodiment 3

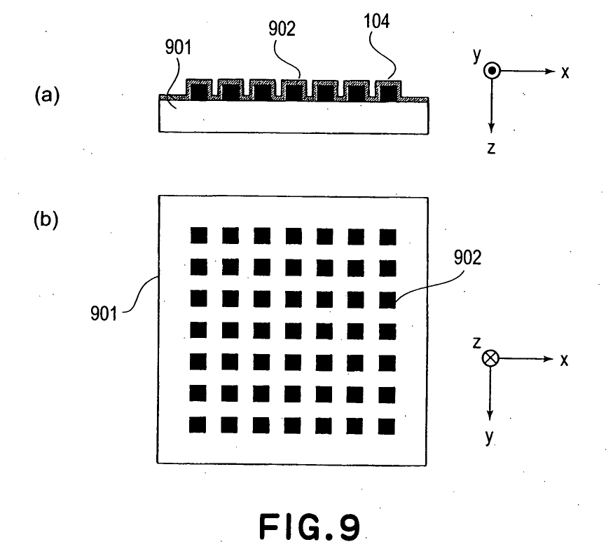

FIGS. 9(a) and 9(b) show a structure of a sensor medium having a pattern array shape in this embodiment, wherein FIG. 9(a) is a sectional view thereof, and FIG. 9(b) is a top view thereof.

As shown in these figures, a fine metal particle array 902 is disposed on a transparent substrate 901 in a two-dimensional direction with a pitch which is substantially equal to a wavelength of SPP wave.

In this embodiment, by using the fine metal particle array instead of the small opening array disposed on the thin metal film, a propagation loss is decreased, so that the SPP wave is propagated farther in an xy plane direction. As a result, an S/N ratio for signal detection can be improved.

A mechanism of propagation of SPP wave in the fine metal particle array may be such that an SPP wave (localized plasmon) propagated along one fine metal particle is scattered at an end portion to excite an SPP wave, propagated along an adjacent fine metal particle, which is also scattered at an end portion and such that an SPP wave propagated along a periphery of one fine metal particle excites an SPP wave propagated along a periphery of an adjacent fine metal particle by an interaction between the one fine metal particle and the adjacent fine metal particle. In this embodiment, a circumferential length of fine metal particle is substantially equal to an integral multiple of a wavelength of SPP wave, so that it becomes possible to increase a strength of SPP wave propagated along the periphery of fine metal particle. For this reason, the propagation of SPP wave on the basis of the latter mechanism is increased.

### Embodiment 4

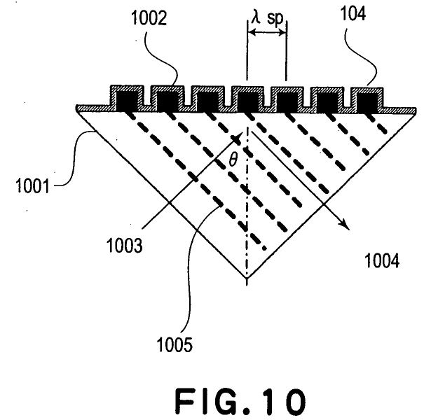

FIG. 10 shows a structure of a sensor medium using a prism 1001 as a substrate in this embodiment.

Referring to FIG. 10, on an upper surface of the prism 1001, a thin metal film 1002 provided with a fine metal particle array, a small opening array or a small uneven structure array, and is covered with a sensor material 1004. In this embodiment, the array of fine metal particle, small opening or small uneven structure has a pitch which is substantially equal to a wavelength λsp of SPP wave.

Such an array structure is irradiated with light from the prism 1001 side.

In order that a spacing, between adjacent wavefronts of incident light 1003, defined by the upper surface of the prism 1001 (a length between adjacent wavefronts along the upper surface of the prism 1001) can be substantially equal to a pitch of the array of fine metal particles, small openings or small uneven structure, a wavelength λ and an incident angle θ of the incident light 1003 are adjusted to satisfy:
λsp≈λ/n·sin θ,
wherein n represents a refractive index of the prism 1001.

By doing so, it becomes possible to effectively excite the SPP wave at the upper surface of the prism 1001 by the incident light 1003.

In this embodiment, detection of a signal may be performed by measuring a spectrum distribution of reflected light 1004 through a spectroscope or by detecting an intensity of reflected light with the use of monochromatic light having at least one wavelength as the incident light. It is also possible to detect a deviation in peak position in an incident angle (θ)-dependent curve of the reflected light intensity by gradually changing the incident angle θ of incident light in a small range. This has a structure similar to that of a surface plasmon resonance (SPR) sensor according to Kretschmann configuration ATR (attenuated total reflectance) method. By the combination of this structure with the periodic structure of SPP wave, high sensitivity detection is realized.

### Embodiment 5

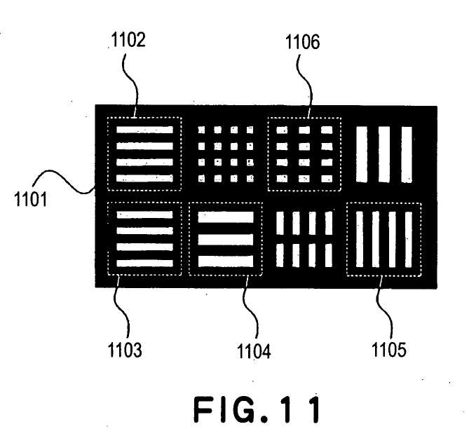

FIG. 11 shows a structure of a multi-channel sensor medium provided with a plurality of slits and two-dimensional small opening arrays 1102 to 1106 in a thin metal film 1101. In this embodiment, a plurality of sets of periodic arrangement of slits or small openings different in size and/or pitch (spacing) of slits or small openings are provided in the thin metal film, and may include a set of periodic arrangement of slits or small openings having the same size and/or pitch.

In the sensor medium shown in FIG. 11, a small opening array A (1102) and a small opening array B (1103) have the same size and the same pitch. Further, each of other small opening arrays C (1104), D (1105), and E (1106) has a size and a pitch which are different from those of the small opening arrays A and B.

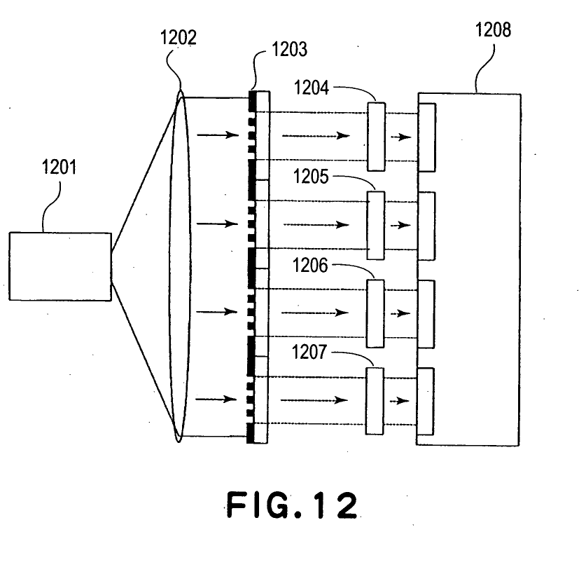

FIG. 12 shows a structure of a multi-channel sensor apparatus including the multi-channel sensor medium shown in FIG. 11. Referring to FIG. 12, light emitted from a tungsten lump 1201 is changed to substantially parallel light by a collimator lens 1202 to enter a multi-channel sensor medium 1203. A plurality of transmitted light fluxes passed through the respective small opening arrays of the multi-channel sensor medium 1203 are caused to enter a CCD (charge-coupled device) camera 1208 through respective filters A (1204), B (1205), C (1206), and D (1207), thus providing transmission pattern information.

In the sensor apparatus, e.g., light fluxes passed through the small opening array A (1102) and the small opening array B (1103) which have the same shaped pattern are caused to pass through band-pass filters having different wavelengths and compared with each other, or the sensor medium is irradiated with two monochromatic lights having different wavelengths to compare resultant transmitted light intensities of the two monochromatic lights, whereby it is possible to obtain relative spectrum information which does not dependent on the irradiated light intensity.

More specifically, as shown in FIG. 3, light intensity signals are detected at two wavelengths λ0 and λ1 which are opposed from each other in shifting direction from a resonance peak position and are compared with each other. As a result, after the sensing reaction, changing directions of the detected signals are opposite from each other, so that it becomes possible to effect higher sensitivity detection under no influence of error factors, such as a change in incident light intensity and a change in incident angle.

Similarly, sensor materials different in kind are disposed in openings of the small opening arrays A (1102) and B (1103) having the same shape pattern and light fluxes passed through the respective small opening arrays are detected independently, whereby it is possible to obtain a plural pieces of sensing information at the same time. Further, by effecting a relative comparison therebetween, it becomes possible to perform high sensitivity detection by differential detection.

In addition, it is possible to obtain different pieces of information simultaneously by comparing patterns or two-dimensional patterns which are different in small opening pitch or arrangement direction. By changing a signal peak position with the use of patterns different in pitch or size, it is possible to arbitrarily select a spectrum range for detecting a signal. As a result, it becomes possible to perform detection at a plurality of light wavelengths, so that it is possible to readily separate a signal by an interference filter or the like even when the multi-channel sensor medium is integrated to have a small size as in this embodiment.

### Embodiment 6

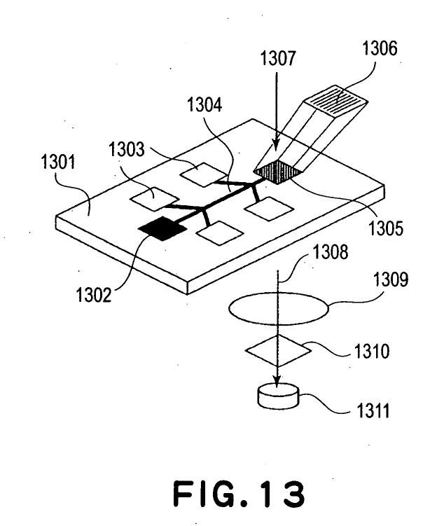

FIG. 13 shows a structure of an integrated sensor medium in a μ-TAS (Micro Total Analysis System, also called “lab-on-a-chip”) in this embodiment.

In a μ-TAS 1301 shown in FIG. 13, a liquid to be tested, injected from a sample liquid injection port 1302, passes through a passage 1304 to react with a reaction liquid injected from a reaction liquid injection port 1303, and then reaches a detection portion 1305. At the detection portion 1305, a small slit opening 1306 comprising fine metal particles, small openings or a small uneven structure for detection on the basis of a principle according to the present invention is provided, as shown in FIG. 13 as an enlarged portion. The liquid to be tested penetrates into the small slit opening 306 and reacts with a sensor material in the fine metal particles, small openings or small uneven structure. The detection portion is irradiated with excited light 1307 to produce fluorescence 1308 through the small slit opening 1306. The fluorescence 1308 is concentrated by a lens 1309, passed through a filter 1310, and is detected by a photomultiplier 1311.

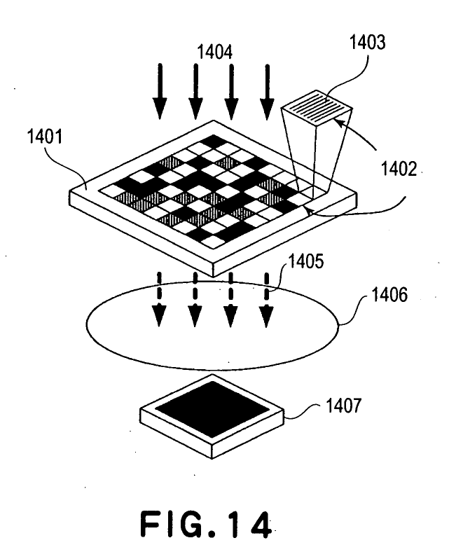

FIG. 14 shows a structure of a sensor medium, as another example in this embodiment, which is integrally supported on a DNA chip or a protein chip.

Referring to FIG. 14, a DNA chip/protein chip 1401 has a plurality of detection cells 1402 provided with an array 1403 of fine metal particles, small openings or a small uneven structure. The array 1403 is provided with a sensor material at its inner side surface. The sensor material is irradiated with excited light 1404 to produce fluorescence 1405. A pattern of the fluorescence 1405 forms an image on a surface of a CCD camera 1407 through a lens 1406, thus providing pattern information.

### INDUSTRIAL APPLICABILITY

As described hereinabove, according to the present invention, a sensor medium employed in the present invention can be used in combination with various sensors, so that a signal intensity is enhanced to permit detection with high accuracy.

1. A chemical sensor for detecting a reaction of a sensor material with a specimen on the basis of an intensity of a surface plasmon polariton wave propagated along a surface of a sensor medium comprising the sensor material, said chemical sensor comprising the sensor medium,
wherein said sensor medium comprises a periodic structure and the sensor material disposed on the periodic structure, the periodic structure having a pitch substantially equal to an integral multiple of a wavelength of the surface plasmon polariton wave generated by irradiating an interface between the periodic structure and the sensor material with light.

2. A sensor according to claim 1, wherein the sensor material is a biochemical sensor material.

3. A sensor according to claim 1, wherein the periodic structure comprises a plurality of openings provided in a metal film with a predetermined pitch, the openings having a size smaller than a wavelength of the irradiation light.

4. A sensor according to claim 3, wherein the openings have a substantially circular shape or a substantially polygonal shape, and their periodic arrangement is a two-dimensional arrangement in the metal film surface.

5. A sensor according to claim 3, wherein the openings have a slit shape, and their periodic arrangement is a one-dimensional arrangement in the metal film surface.

6. A sensor according to claim 5, wherein the openings include adjacent two openings sandwiching a metal film portion having a length of circumferences which is a substantially integral multiple of a wavelength of the surface plasmon polariton wave.

7. A sensor according to claim 3, wherein the periodic structure is provided in a plurality of periodic structures, which have the same or different sizes and/or pitches of their openings and the same or different arrangement directions.

8. A sensor according to claim 1, wherein the periodic structure comprises at least one opening provided in a metal film with a predetermined pitch and at least one recess portion or projection portion provided in the metal film, the opening having a size which is smaller than a wavelength of the irradiation light.

9. A sensor according to claim 8, wherein the opening and the recess portion or the projection portion have a substantially circular shape or a substantially polygonal shape, and their periodic arrangements are a two-dimensional arrangement.

10. A sensor according to claim 9, wherein the two-dimensional arrangement is such an arrangement that the recess portion or the projection portion is disposed concentrically around the opening.

11. A sensor according to claim 8, wherein the opening and the recess portion or the projection portion have a slit shape, and their periodic arrangements are a one-dimensional arrangement.

12. A sensor according to claim 8, wherein the opening includes adjacent two openings sandwiching a metal film portion having a length of circumference which is a substantially integral multiple of the wavelength of the surface plasmon polariton wave.

13. A sensor according to claim 3, wherein the metal film is a film of a metal or alloy selected from the group consisting of gold, silver, copper, and aluminum.

14. A sensor according to claim 1, wherein the periodic structure comprises fine metal particles disposed on a substrate with a predetermined pitch, the fine metal particles having a size which is smaller than the wavelength of the surface plasmon polariton wave.

15. A sensor according to claim 14, wherein the fine metal particles have a length of circumference which is a substantially integral multiple of the wavelength of the surface plasmon polariton wave.

16. A sensor according to claim 1, wherein the sensor medium comprises the periodic structure and a substrate for the sensor material disposed on the periodic structure, the substrate comprising a prism.

17. A sensor apparatus, comprising:
a chemical sensor according to claim 1,
a light source for irradiating the chemical sensor with light, and
a photodetector for detecting light transmitted through or reflected from the chemical sensor.

18. An apparatus according to claim 17, wherein the photodetector comprises a spectroscope.

19. An apparatus according to claim 18, wherein the photodetector comprises means for detecting light transmitted through a band-pass filter.

20. An apparatus according to claim 17, wherein the sensor medium is integrally supported in a micro total analysis system prepared through a semiconductor process.

21. An apparatus according to claim 17, wherein the sensor medium is integrally supported in a DNA chip prepared through a semiconductor process.

22. An apparatus according to claim 17, wherein the sensor medium is integrally supported in a protein chip prepared through a semiconductor process.
